(19) United States
(12) Patent Application Publication
(10) Pub. No.: US 2006/0193750 A1
(43) Pub. Date: Aug. 31, 2006

(54) Electrochemical detection of explosives in air

(75) Inventors: Boris Filanovsky, Jerusalem (IL); Alexander Sakin, Jerusalem (IL); Jacob Hiterer, Jerusalem (IL)

Correspondence Address: C/o Bill Polkinghorn;DR. MARK FRIEDMAN LTD., 9003 Florin Way, Upper Marlboro, MD, 20772, US

(73) Assignee: MEDIS EL LTD.

(21) Appl. No.: 11/066,213

(22) Filed: Feb. 28, 2005

(51) Int. Cl. G01N 33/00

(52) U.S. Cl. 422/94; 422/88

(57) ABSTRACT

A portable device for detecting an explosive substance present in air, the device including: (a) a mechanism for drawing an air sample into the device; (b) a solid trapping material having a surface for trapping explosive particles in the air sample; (c) a collector for containing a solvent, the collector associated with the surface, the solvent for producing dissolved explosive material by: (i) removing and dissolving the portion of particles from the surface, and (ii) directly dissolving a remainder of the particles of the explosive material, (d) an electrode unit, associated with the collector, for producing a signal corresponding to a presence of the dissolved explosive material, and (e) circuitry for determining the presence of the dissolved explosive material based on the signal produced by the electrode unit.

### FIELD AND BACKGROUND OF THE INVENTION

The present invention relates to a method of, and device for, detecting trace amounts of explosives in air, and more particularly, to an electrochemical method of, and portable electrochemical device for, detecting nitrates in air.

Due to heightened terrorist activities throughout the world, there exists an acute need to safeguard transportation, facilities and other locales that are vital to the public safety and to government functions. To address the need, inventors have concentrated on developing portable instruments for detection of explosives in the air surrounding a suspect object. One of the practical applications of such portable device is the inspection of facilities suspected of producing and/or storing explosives. The air in these facilities usually contains vapors of the explosives that otherwise can be detected only by the canine sense of smell.

Due to heightened terrorist activities throughout the world, there exists an acute need to safeguard transportation, facilities and other locales that are vital to the public safety and to government functions. Consequently, there is an increased interest in portable units for detecting explosive materials, including nitro-aromatic compounds such as 2,4,6-trinitrotoluene (TNT), dinitrotoluene (DNT), and similar derivatives.

Many detection methods have been used to detect explosive materials. These methods include gas and HPLC chromatography, x-ray scattering, neutron analysis, nuclear quadrupole resonance, and mass spectrometry (U.S. Pat. No. 6,571,649). These methods generally require expensive and sophisticated equipment, (e.g., high vacuum), equipment that is not portable (e.g., cylinders of compressed gases), and/or have a complicated sample preparation. These techniques, are therefore, not appropriate for low cost portable field-testing for trace explosive materials. Some of these methods for explosives detection have been reviewed in “Explosives detection systems (EDS) for aviation security” (Singh, S., Signal Processing vol. 83, 2003, p. 31-55).

Another known method for the detection of trace amounts of explosive materials utilizes immunochemical sensors. For example, U.S. Pat. No. 6,573,107 is directed towards the immunochemical detection of explosive substances in the gas phase using surface plasmon resonance spectroscopy. Immunochemical detection methods potentially offer high selectivity and high sensitivity.

Electrochemical detection refers to the use of electrodes, immersed in an electrolyte, and connected to an instrument that varies the voltage applied to the electrodes. The instrument measures the current flow between the electrodes. Typically, the electrode potential is varied; and an electric current flows between the electrodes that is characteristic of the presence of electrochemical active substances in the electrolyte. The magnitude of the current is proportional to the concentration of the electrochemically-active substances. It is well known that TNT and other nitro-aromatic compounds are reduced electrochemically at the cathode and may be detected by electrochemical detection. Wang, et. al. (Analytica Chimica Acta, vol. 485 (2003), p. 139-144), teach the monitoring of TNT in natural waters using an electrochemical technique. Wang, et. al., report a measurement sensitivity of 0.003 μA/ppb of TNT in natural seawater. This sensitivity level is achieved by subtracting the background signal, in natural seawater not contaminated by TNT, caused by the reduction of dissolved oxygen. One of the instant inventors, Boris Filanovsky, has taught the use of carbon/Hg film electrode materials in an aqueous solvent (Reviews Analytical Chemistry, vol. 18, no. 5, 1999, p. 293). This electrode material enables the background signal to be reduced by separating the atmospheric O₂ background current from the TNT current. However, the sensitivity reported is only ˜0.7 μA/μM (˜0.003 μA/ppb), which is comparable to the sensitivity reported by Wang, et. al.

U.S. patent application Ser. No. 10/715,489, which is not to be construed as prior art with respect to the present invention, teaches an electrochemical method and sensor for the detection of traces of explosives, specifically, for the detection of trace amounts of nitro-aromatic compounds including TNT (and similar substances) in air. The measurement of nitro-aromatic compounds requires only about 30-40 sec, which represents an improvement over much of the prior art.

Despite these positive developments in the prior art, there remains considerable room for improvement in achieving a practical portable field test for trace explosive materials. For example, it would be highly advantageous for such a portable field to use a minute quantity of solvent for each test, and to achieve facile regeneration of the solvent in-situ.

Another area that is in clear need of improvement is the pre-concentration of the trace explosive material. The task of detecting explosives in air requires overcoming the problem of extremely low concentrations of the explosive vapors, often at levels of 1 ppb. To address this problem, attempts have been made to concentrate the vapors by heating and trapping the vapors in a highly absorbent material such as glass wool. However, such highly absorbent materials typically absorb the explosive powder in a substantially irreversible fashion, and must be frequently replaced, making such materials impractical for portable detection units.

In an attempt to concentrate and detect trace quantities of explosives, U.S. Pat. Nos. 5,092,218 and 5,123,274 to Fine, et al., and to Carroll, et al., respectively, teach a method of stripping vapors from surfaces using heat and suction from a hand-held sample gun. The vapors are collected on a surface coated with a gas chromatograph material that traps explosive vapors, but repels nitric oxide. The vapors are subsequently concentrated. A high-speed gas chromatograph separates the vapors, after which, specific vapors are decomposed in two pyrolyzers arranged in parallel and the nitric oxide resulting therefrom is detected. A low-temperature pyrolyzer containing silver produces NO from nitroamines or nitrite esters; a high-temperature pyrolyzer decomposes all explosives vapors to permit detection of the remaining explosives. Also disclosed is a series arrangement of pyrolyzers and gas chromatographs.

Though increasing sensitivity of the method by increasing vapor concentration due to sampling air at an elevated temperature, the method requires the use of complicated temperature controls to avoid destruction of the explosive trace compounds by excessive heat, and to avoid a drop in detection sensitivity due to too low a temperature.

Several deficiencies are apparent in the teachings of U.S. Pat. Nos. 5,092,218 and 5,123,274. The use of a pyrolyzer as well as a gas chromatograph calls for complicated procedures required to validate the specific elution times for each of the suspect explosives, to calibrate the baseline of the chromatograph and the pyrolyzer, and to optimize the signal-to-noise ratio that is critical to reliability of chromatographic results.

The use of hydrogen as a carrier gas, unique collectors and concentrators, high-speed heaters, NO detectors, and a temperature-programmble gas chromatograph renders the disclosed prior-art devices unusually expensive and complicated.

There are also significant dangers inherent in the methods taught by the above patents. The heating of a suspect surface may trigger an explosion if explosives are actually located beneath the suspect surface. Furthermore, the highly explosive characteristics of hydrogen gas used in the gas chromatograph only add to the hazards of employing heating elements within confined and often unventilated locations common to explosives detection situations. The use of heating elements, pyrolyzers, a gas chromatograph and hydrogen requires that the personnel operating the device is safeguarded by additional features that render the device heavy and cumbersome, and cause undue delays while the device is readied for the next sampling.

There is, therefore, a recognized need for, and it would be highly advantageous to have, a device for and method of concentrating and detecting explosives vapors that is safe, simple and sensitive. It would be of further advantage to have a device that is portable and easy to operate, provides quick and accurate results and has a short turnaround period between samplings.

### SUMMARY OF THE INVENTION

The present invention is a portable device for detecting explosives in air incorporating an inventive air sampler and a chemically modified electrochemical sensor.

According to the teachings of the present invention there is provided a portable device for detecting at least one explosive substance present in air, the device including: (a) a mechanism for drawing an air sample into the device; (b) a solid trapping material having a surface for trapping a portion of particles of an explosive material in the air sample; (c) a collector for containing a solvent, the collector associated with the surface, the solvent for producing dissolved explosive material by: (i) removing and, dissolving the portion of particles from the surface, and (ii) directly dissolving a remainder of the particles of the explosive material, (d) an electrode unit, associated with the collector, for producing a signal corresponding to a presence of the dissolved explosive material, and (e) circuitry for determining the presence of the dissolved explosive material based on the signal produced by the electrode unit.

According to further features in the described preferred embodiments, the trapping material is a reversibly trapping material.

According to still further features in the described preferred embodiments, the trapping material includes a material selected from the group of materials consisting of polytetrafluoroethylene, cross-linked polyethylene, and polypropylene.

According to still further features in the described preferred embodiments, the trap is associated with the collector by direct fluid communication.

According to still further features in the described preferred embodiments, the device further includes an electrochemical mechanism, designed and configured to be in fluid communication with the collector, upon demand, for electrochemically regenerating the solvent.

According to still further features in the described preferred embodiments, the device further includes a control device for enabling fluid communication between the electrochemical mechanism and the collector, upon demand.

According to still further features in the described preferred embodiments, the surface of the solid trapping material has a reversibility factor (R), characterized by:

$$Reversibility Factor ( R ) = \frac{Weight of explosive material removed}{( Volume of Solvent ) \times ( Time )}$$

wherein the explosive material is trinitrotoluene, the solvent is ethylene glycol, the weight is measured in micrograms, the volume is measured in milliliters, and the time is measured in seconds, and wherein R is at least 0.010.

According to still further features in the described preferred embodiments, R is at least 0.03, preferably at least 0.05, more preferably at least 0.10, and most preferably at least 0.12.

According to still further features in the described preferred embodiments, the solvent includes a solvent selected from the group of solvents consisting of ethylene glycol, propylene glycol, diethylene glycol dimethyl ether, triethylene glycol dimethyl ether, alcohols having a carbon chain length of 2 to 3, and mixtures thereof.

According to still further features in the described preferred embodiments, the electrode unit includes an electrode material modified by treatment thereof with at least one chemical modifier that increases electron transfer kinetics of nitro-aromatic compounds.

According to still further features in the described preferred embodiments, the at least one chemical modifier includes an aromatic organic compound.

According to still further features in the described preferred embodiments, the at least one chemical modifier includes a compound selected from the group of compounds consisting of amine, sulfanilamide, amino-naphthalene and aromatic derivatives thereof, and a para-aminobenzenesulfonylamine having the structure NH₂—C₆H₄—SO₂—NR′R″, wherein the amine has R′ and R″ moieties selected from the group of radicals consisting of H, X, CH₃, CH₂X, and CHX₂, where H is hydrogen and X is a halogen or a halogen-containing moiety.

According to still further features in the described preferred embodiments, the at least one chemical modifier includes a compound selected from the group of compounds consisting of amino-aromatic compounds, alkyl-aniline compounds, halide derivatives of alkyl aniline compounds and hydroxyl-aniline compounds.

According to still further features in the described preferred embodiments, the at least one chemical modifier includes a compound selected from the group of compounds consisting of phenylene-diamine, diphenylene-diamine, and diphenylene-triamine.

According to still further features in the described preferred embodiments, the at least one chemical modifier includes aniline.

According to still further features in the described preferred embodiments, the electrode unit includes an electrode material selected from the group consisting of carbon and gold.

According to still further features in the described preferred embodiments, the electrode unit includes an electrode material modified by treatment thereof with at least one chemical modifier, so as to produce, in a presence of the explosive material in the solvent, a current peak, the peak occurring within a potential range of 0.0 to minus 1.1 Volts.

According to still further features in the described preferred embodiments, the current peak occurs within a potential range of minus 0.4 to minus 0.65 Volts.

According to still further features in the described preferred embodiments, the id current peak occurs within a potential range of minus 0.48 to minus 0.60 Volts.

According to still further features in the described preferred embodiments, the circuitry includes a direct current pulse generator for registering at least 2 potential steps, wherein the duration of each of the potential steps is in a range of 0.1 to 2.5 seconds.

According to still further features in the described preferred embodiments, the potential steps are in a cathode range that occurs minus 200 to minus 250 millivolts after a peak potential.

According to another aspect of the present invention there is provided a portable device for detecting at least one explosive substance present in air, the device including: (a) a mechanism for drawing an air sample into the device; (b) a solid trapping material having a surface for trapping a portion of particles of an explosive material in the air sample; (c) a collector for containing a solvent, the collector associated with the surface, the solvent for producing dissolved explosive material by removal of the portion of particles from the surface; (d) a contacting mechanism for contacting the solvent with the surface so as to effect the removal; (d) an electrode unit, associated with the collector, for producing a signal corresponding to a presence of the dissolved explosive material, and (e) circuitry for determining the presence of the dissolved explosive material based on the signal produced by the electrode unit.

According to yet another aspect of the present invention there is provided a method for detecting at least one explosive substance in air, the method including the steps of: (a) providing a device including: (i) a mechanism for drawing an air sample into the device; (ii) a solid trapping material having a surface for trapping particles of an explosive material in the air sample; (iii) a solvent for removing the particles from the surface and for dissolving the particles; (iv) a collector for receiving the solvent, the collector associated with the surface, and (v) an electrode unit, associated with the collector, for producing a signal corresponding to a presence of a dissolved explosive material; (b) drawing an air sample into the device using the mechanism, so as to trap the particles on the surface; (c) removing the particles from the surface into the solvent; (d) dissolving the particles in the solvent to produce the dissolved explosive material, and (e) producing the signal corresponding to the presence of the dissolved explosive material.

According to further features in the described preferred embodiments, the method further includes: (f) determining, automatically, the presence of the dissolved explosive material based on the signal produced by the electrode unit.

According to still further features in the described preferred embodiments, the removing of the particles from the surface into the solvent is performed by automatically circulating the solvent, upon demand, so as to fluidly contact the surface.

According to still further features in the described preferred embodiments, the method further includes: (f) regenerating the solvent within the device.

According to still further features in the described preferred embodiments, the regenerating is performed using regeneration electrodes.

### BRIEF DESCRIPTION OF THE DRAWINGS

The invention is herein described, by way of example only, with reference is to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1A is a conceptual diagram showing one embodiment of the method of the present invention;

FIG. 1B is a schematic diagram showing an inventive collector having a reversibly trapping surface and a detector, according to one embodiment of the present invention;

FIG. 1C is a schematic diagram of the device of the present invention;

FIG. 2 is a conceptual diagram of the detection circuitry and a collector of the inventive device;

FIG. 2A is a graph of time vs. electric potential showing a square wave applied to a detector for detecting explosive materials;

FIG. 3 is a graph showing the measurement sensitivity of an electrode to the presence of TNT, according to the present invention;

FIG. 3A is a graph based on FIG. 3, in which the background signal has been subtracted from the response curves;

FIG. 4 is a graph of current vs. time showing a first current versus time cycle during a pulse measurement, and

FIG. 5 is a graph of potential vs. change in current illustrating the dependence of an analytical signal on the particular type of chemical modifier.

### DESCRIPTION OF THE PREFERRED EMBODIMENTS

The present invention is a portable device for detecting explosives in air incorporating an inventive air sampler and a chemically modified electrochemical sensor.

The principles and operation of the device according to the present invention may be better understood with reference to the drawings and the accompanying description.

Before explaining at least one embodiment of the invention in detail, it is to be understood that the invention is not limited in its application to the details of construction and the arrangement of the components set forth in the following description or illustrated in the drawing. The invention is capable of other embodiments or of being practiced or carried out in various ways. Also, it is to be understood that the phraseology and terminology employed herein is for the purpose of description and should not be regarded as limiting.

Traditionally, high-performance trapping materials such as glass wool have been used to trap trace materials in air samples. However, such materials typically trap the explosive powder in a substantially irreversible fashion, and must be frequently replaced, making such materials impractical for portable detection units.

Surprisingly, the inventors have discovered that various materials that are widely considered to be “poor” trapping materials, such as polytetrafluoroethylene (Teflon™), cross-linked polyethylene, and/or polypropylene, are particularly suitable for use in the device and method of the present invention. The physical and chemical association of the explosive substances with these materials is such that the explosive materials are readily flushed and liberated from the surface. Thus, almost paradoxically, the materials that are widely considered to be “poor” trapping materials have been found to be the most effective for portable detection units for use in the field, for which the number of samples that can be processed without replacement of materials is a parameter of cardinal importance.

As used herein in the specification and in the claims section that follows, the term “trapping” refers to a process of causing particulate matter to adhere to, or to be retained by a surface, and the term “trapping” also refers to a process where vapors of a substance are condensed on or adsorbed on a surface.

As used herein in the specification and in the claims section that follows, the term “nominal surface area” with regard to a trapping surface, refers to a surface area of the trapping surface based on the global geometric dimensions, without regard to microstructure. Hence, a cylinder having an inner diameter of 2 cm and a length of 10 cm has a nominal surface area of πDL=20π cm².

As used herein in the specification and in the claims section that follows, the term “effective surface area” with regard to a trapping surface, refers to the microstructural area of the trapping. Hence, a cylinder having an inner diameter of 2 cm and a length of 10 cm, and a ratio of effective surface area to nominal surface area of 3, has an effective surface area of 3πDL=60π cm².

As used herein in the specification and in the claims section that follows, the term “reversibility”, “reversible”, “reversibly”, and the like, with respect to a trap surface material, refers to a characteristic of a material to easily assume a prior state. More particularly, the reversibility is defined as a structural characteristic of a surface having a particular surface area for trapping a standard, particulate explosive material (TNT), such that at least 90% of the particulate explosive material adhering to it is removed by flushing, at room temperature, within a period of time period not exceeding 20 seconds. In quantitative terms, a reversibly-trapping surface meets the following reversibility criterion:

$$Reversibility Factor ( R ) = \frac{( Weight of explosive material removed )}{( Volume of Solvent ) \times ( Time )}$$

wherein R≧than 0.010 [expressed in μg/(ml solvent×seconds)]. Preferably, R is at least 0.03, more preferably, at least 0.05, still more preferably, at least 0.10, and most preferably, at least 0.12.

Thus, given a trap surface material initially having 5.0 μg of TNT particles adhering thereto, and a flushing action resulting in 90% of the TNT particles adhering thereto are removed within 5 seconds using 10 ml of ethylene glycol solvent,
R=4.5 μg/(10 ml×5 seconds)=0.09.
This trap surface material satisfies the reversibility criterion defined hereinabove, such that the trap surface material is a reversibly-trapping material.

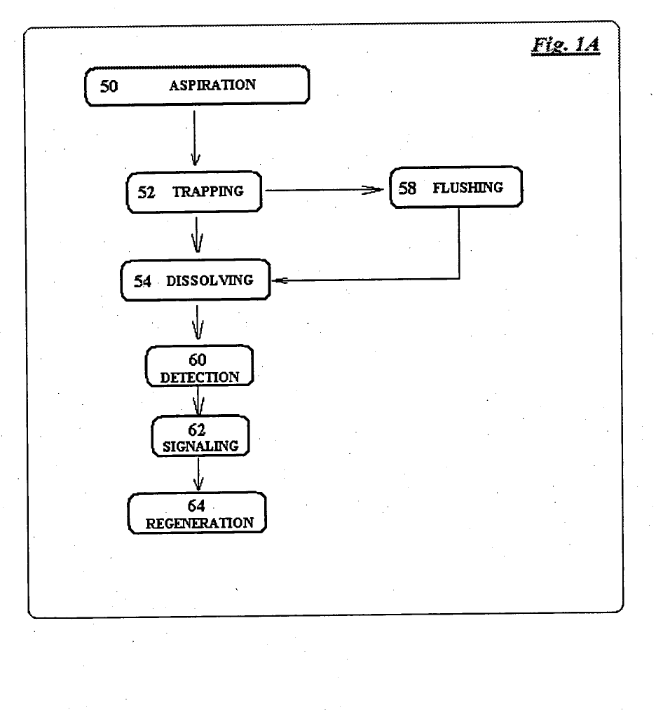

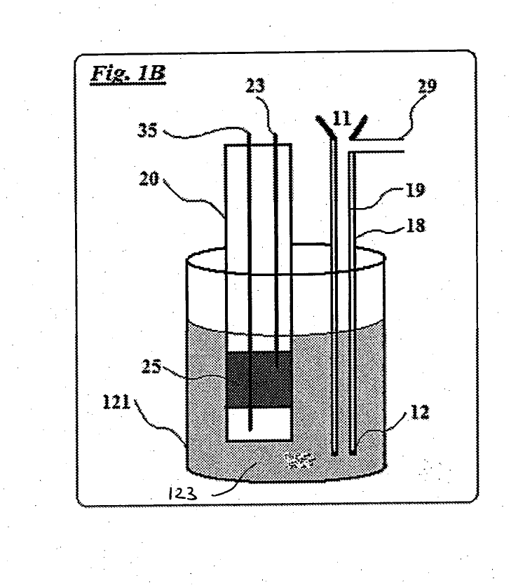

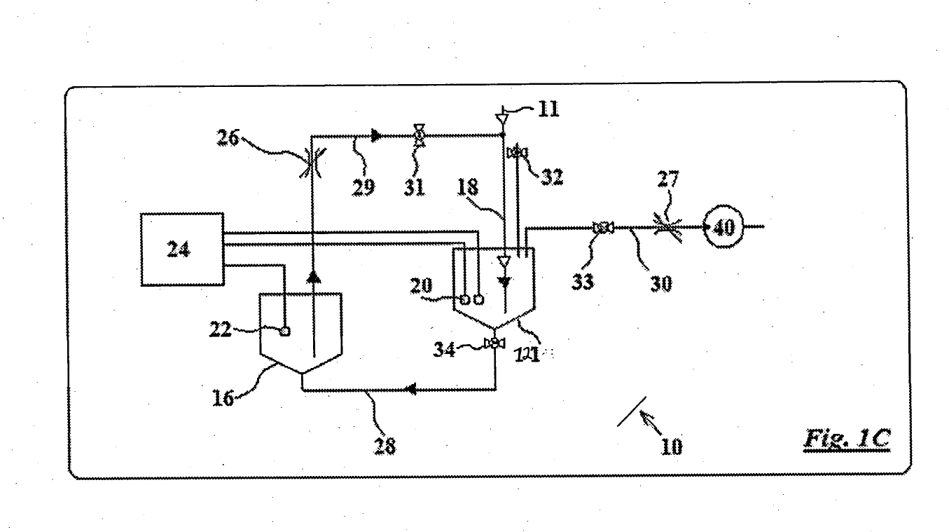

The collection, concentration and detection of explosive materials in air, in one embodiment of the present invention, is illustrated conceptually in FIG. 1A. Air to be tested is aspirated into the device in step 50. In step 52, explosive particles from the air sample reversibly adhere to the surface of the trap.

After the trapping operation, the vapors and particles remaining in the air stream are dissolved in the solvent within a collector (step 54). In step 58, an additional amount of the solvent is used to flush the trap in order to rinse any explosive particles adhering to the surface of the trap into the collector. Dissolution of the flushed particles is completed in the solvent within the collector in step 54. In step 60, the liquid, which contains the dissolved explosives from step 54, is subjected to electrochemical analysis and detection. Nitrates contained in the explosive vapors react with sulfanilamide or other moieties of the carbon matrix of the detector of the device, thereby being reduced to amines and causing a change in the electric potential between the reference electrode and the detection electrode. The change generates a signal that is amplified and announced as an alarm in step 62. The solvent is then regenerated (step 64), so as to prepare the device for another sampling.

As used herein in the specification and in the claims section that follows, the term “regenerating” refers to a process of removing contaminants and restoring properties of a substance, wherein the properties include chemical, physical and electrochemical properties.

A schematic illustration of the device of the present invention is shown in FIG. 1B. An air sample is introduced through an air inlet 11. Particles of explosive material in the air sample are trapped by a reversibly trapping surface 19 of a particle trap 18. The rest of the air sample is introduced to solvent 123, which is disposed in a hermetically-sealed collector 121. Regenerated solvent, which is used to flush reversibly trapping surface 19, is delivered to collector 121 via a solvent line 29.

The detection of explosive materials takes place in a detector 20, which is immersed in solvent 123. Also immersed in solvent 123 are a lower end 12 of trap 18, and a detector 20 having an analytical electrode 23, a reference electrode 35 and a carbon matrix 25. The carbon matrix, which is preferably modified with sulfanilamide, can be carbon paper, carbon cloth and related materials that are 10-90% porous, and, preferably, 40-60% porous.

Electrodes 23 and 35 are preferably disposable electrodes, and require replacement after a certain number of detection cycles, not less than 100 cycles. Typically, such disposable electrodes, used in conjunction with the present invention, require replacement after about 2 weeks of intensive work.

As described in co-pending U.S. patent application Ser. No. 10/715,489, which is incorporated by reference for all purposes as if fully set forth herein, the modifier molecule is preferably a polar aromatic amine, and more preferably, the modifier molecule is a molecule whose dipole has the most electron-poor cationic, or amine group, which, in a para position, has a most electron-rich group. In one embodiment of the present invention, the electron-rich group is SO₂. A para amine group can include radicals having a N-(R₁, R₂) configuration. The radical R₁ can be of the CₙHₘ formula, where n is 1, inclusive, to 4, inclusive, and m is 3, 5, 7, or 9, wherein m corresponds to n sequentially. The radical R₂ can be of the Cₙ₁ Hₘ₁ formula, where n₁ is 1, inclusive, to 4, inclusive, and ml is 3, 5, 7, or 9, and a modifier molecule can have a radical in a combination wherein n=4 and n₁=1.

For the desired maximum dipole effect in the a para amine group, electro-negative groups can be selected according to a N-(R₃,R₄) configuration. The R₃ and R₄ moieties can be of an —N—R—X formula, where X is a halogen, such as chlorine (Cl), bromine (Br), iodine (I), or fluorine (F), or an oxide thereof, such as chlorate (ClO₄) and related groups. The R₃ and R₄ moieties need not be identical. The most preferred modifier groups are:
1. para-sulfoamine-aniline, NH₂—C₆H₄—SO₂—NH₂,
2. para-sulfo-dimethylamino-aniline, NH₂—C₆H₄—SO₂—N—(CH₃)₂, and
3. para-sulfo-dimethylenebromide-amino-aniline, NH₂—C₆H₄—SO₂—N—(CH₂Br)₂.

An inventive device 10 for detection of explosive materials in air, shown in FIG. 1C, includes collector 121, trap 18, a solvent regeneration vessel 16 and electronic circuitry 24. Detector 20 is disposed in collector 121. Vessel 16 contains regeneration electrodes 22, for regenerating used/contaminated solvent. Vessel 16 communicates fluidly with collector 121 via a solvent line 28 for receiving contaminated solvent from collector 121, and via a solvent line 29 for delivering regenerated solvent to collector 121.

Regeneration electrodes 22 may be bare carbon-cloth electrodes or carbon-paper electrodes.

The flow of solvent through the solvent lines is accomplished by means of a vacuum pump 40, a flow regulator 26, and valves 31, 32, 33 and 34. Vacuum pump 40 communicates with collector 121 via an air line 30, and the air flow therein is regulated by a flow regulator 27.

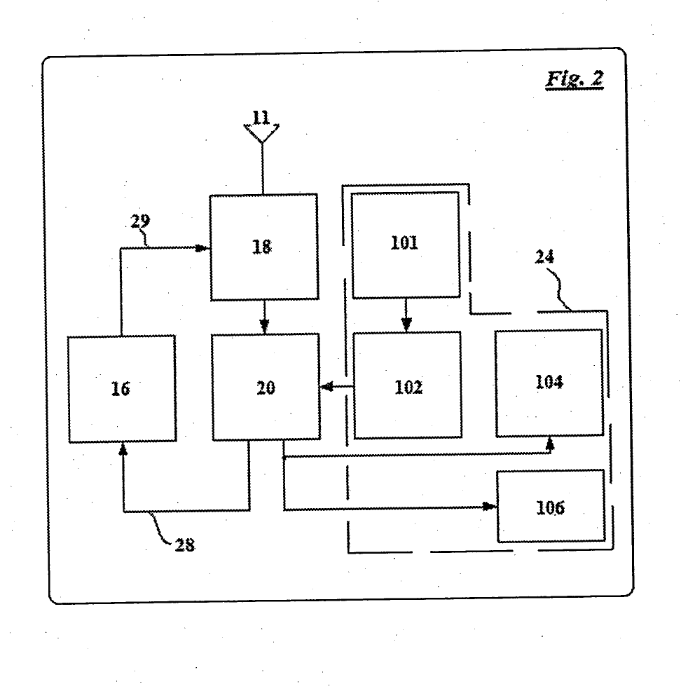

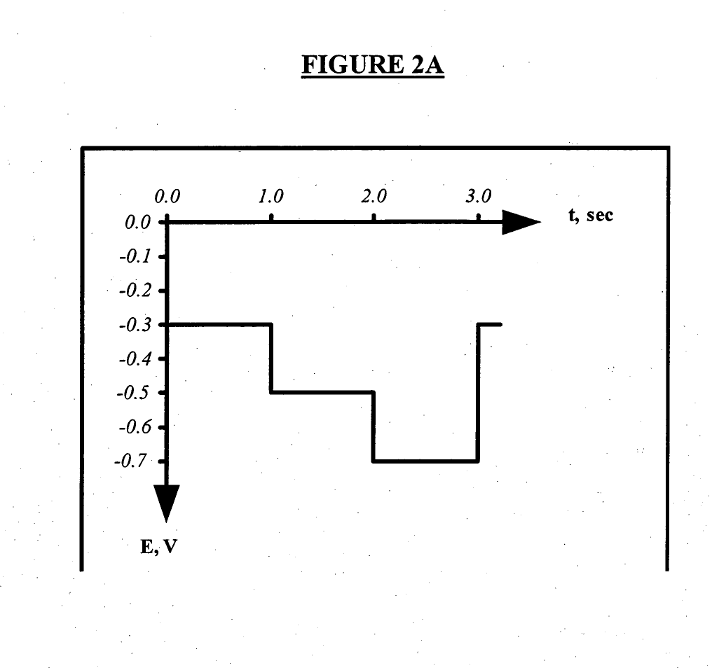

Electronic circuitry 24 (shown in greater detail in FIG. 2 hereinbelow) generates square wave pulses, which, when applied to analytical electrode 23 (see FIG. 1B) in detector 20, cause a change in an electric current and a change in the analytic potential between analytical electrode 23 and reference electrode 35. The current response is a linear function of the concentration of the explosive.

A detection stage of the device operation, as illustrated in FIG. 1C, starts with collector 121 containing a minimal amount (typically 3-10 ml, preferably less than 5 ml) of solvent. Regeneration vessel 16 contains up to 200 milliliters of fresh solvent. An automatic sequence is initiated by circuitry 24, wherein valves 31, 32 and 34 are closed, valve 33 is opened, vacuum pump 40 is activated, air inlet 11 is opened and air is aspirated through inlet 11 of trap 18 for less than 10 seconds. Flow regulator 27 is set such that vacuum pump 40 delivers an air flow within a suitable range of linear velocities. Preferably, the linear velocity should be low enough to trap an appreciable amount of explosive material on the surface of trap 18, but high enough to enable a reasonable sampling time.

After the aspiration stage, valve 31 is opened and inlet 11 is closed. At this time, approximately 5 milliliters of solvent are drawn through solvent line 29 to the reversibly trapping surface of trap 18, typically in a period of 3-5 seconds. Flow regulator 26 is set to achieve a flow rate that is suitable for flushing any explosive material adhering to the surface of trap 18. After flushing of the trap concludes, valve 33 is closed and an analytical step, corresponding to step 60 in FIG. 1B, commences. The analytical step preferably lasts 1-6 seconds, more preferably, 1-2 seconds, and most preferably, less than 1.5 seconds. After the detection, valves 32 and 34 are opened and the solvent in collector 121, containing the dissolved explosive material, is aspirated to regeneration vessel 16 for cleaning and regeneration. After regeneration of the solvent in vessel 16, valves 31, 32 and 34 are closed, valve 33 is opened, and inlet 11 is ready to be opened for the next detection cycle.

A typical operation cycle includes sampling, detection and regeneration stages. The sampling stage lasts typically 10-20 seconds. The detection stage lasts typically 1-2 seconds. The regeneration stage may be substantially continuous, such that the detection and regeneration stages operate concurrently.

FIG. 2 is a conceptual diagram showing communication of electronic circuitry 24 with detector 20. Circuitry 24 contains a DC power supply 101, a controller 102, a display 104, and an alarm 106.

During an analytical cycle, controller 102 applies a square wave 112 (illustrated in FIG. 2A) to detector 20 (specifically, to analytical electrode 23 shown in FIG. 1B). The square wave also corresponds to a change in a potential. The potential of −0.5V is an analytical (reduction) potential, while the potentials of −0.3V and of −0.7V are reference potentials. The current varies linearly with reaction products and also depends on changes in background potential. However, at voltages of −0.3V and −0.7V, the current is affected solely by background potential changes.

The circulation of solvent from detector 20 to regeneration vessel 16 via solvent line 28 and from regeneration vessel 16 to trap 18 via solvent line 29 have been described hereinabove.

### EXAMPLES

Reference is now made to the following examples, which together with the above descriptions, illustrate the invention in a non-limiting fashion.

### Example 1

Air Sampling and Pre-Concentration Test

About 200 mg of trinitrotoluene (TNT) in a paper packet was placed at a distance of 50-60 cm from a sensor element, i.e., approximately 5-10 cm from the end of an air sampler tube of the inventive device. The solvent consisted of a mixture of ethylene glycol and water (4:1 on a molar basis), containing 0.1 M KCl at a pH of 9.5, adjusted with KOH. The analytical electrode was based on a carbon paper matrix and had dimensions of 5×10×0.17 mm. After 30 minutes of anodic polarization at 1.1 V in 1 M H₂SO₄, the analytical electrode was rinsed to pH 7 and chemically modified by soaking the electrode for 40 min. in a 4% solution of sulfanilamide in dimethylsulfoxide (DMSO).

The voltage vs. current curves registered have the following square wave voltammetry:
Eₛₜₐᵣₜ=−0.2 V;
E_{fin}=0.70 V; (Ag/AgCl, 3,5 M KCl);
Square wave amplitude 50 mV;
Square wave frequency=5.0 Hz;
step E=4 mV; (as measured by an Epsilon potentiostat, Bioanalytical Systems Corporation, West Lafayette, Ind., USA)

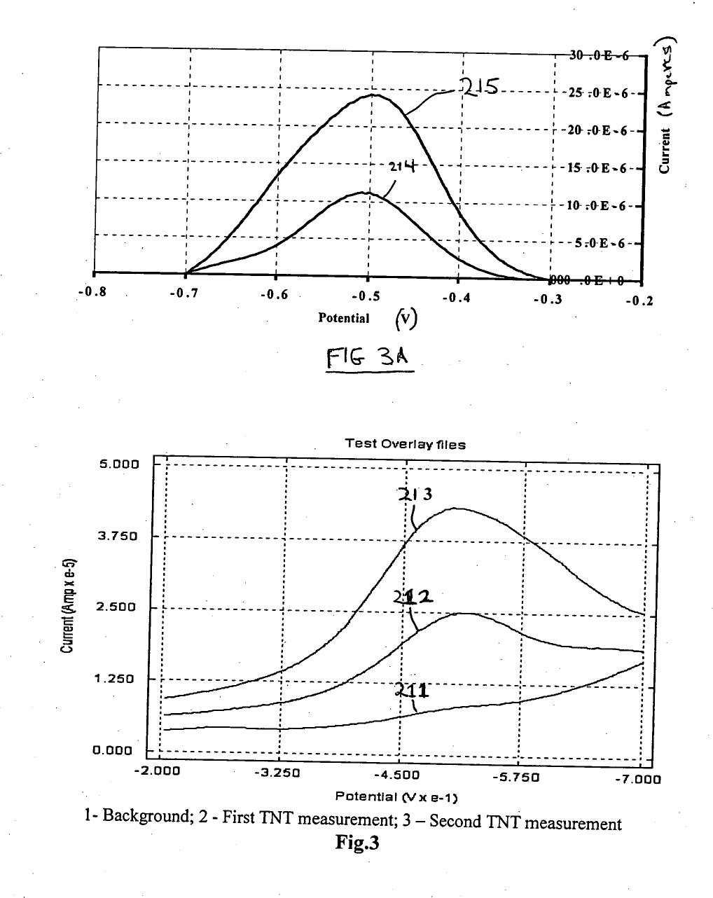

The measurement cycle was carried out according to the following steps:
1. A background current, (represented by curve 211 in FIG. 3) was measured using the chemically modified electrode;
2. Air was sampled through an air sampler for 30 seconds using a vacuum pump having an output of 1.5 liter/minute;
3. The collected explosives were washed off the inner surface (made of polytetrafluoroethylene having a nominal surface area of about 63 cm²) of the sampling tube with 5-7 ml of ethylene glycol and buffer solution (1:1 v/v, pH 9). The weight of the collected explosive particles was approximately 6 μg.

A curve 211 in FIG. 3 is registered in the absence of TNT and curves 212 and 213 is registered in the presence of TNT. Curve 212 corresponds to sampling air for 30 seconds, followed by step 3, and curve 213 to sampling air a second time for 30 seconds, followed by step 3.

Curve 214, shown in FIG. 3A, is curve 212, after subtracting background curve 211; curve 215 is curve 213, after subtracting background curve 211. Both curve 214 and curve 215 display an increase in analytical signal of about 12 μA.

These experimental results demonstrate that the method and device of the present invention enable the facile detection of explosive substances at a distance of 0.6 meters at room temperature.

### Example 2

Detector Test

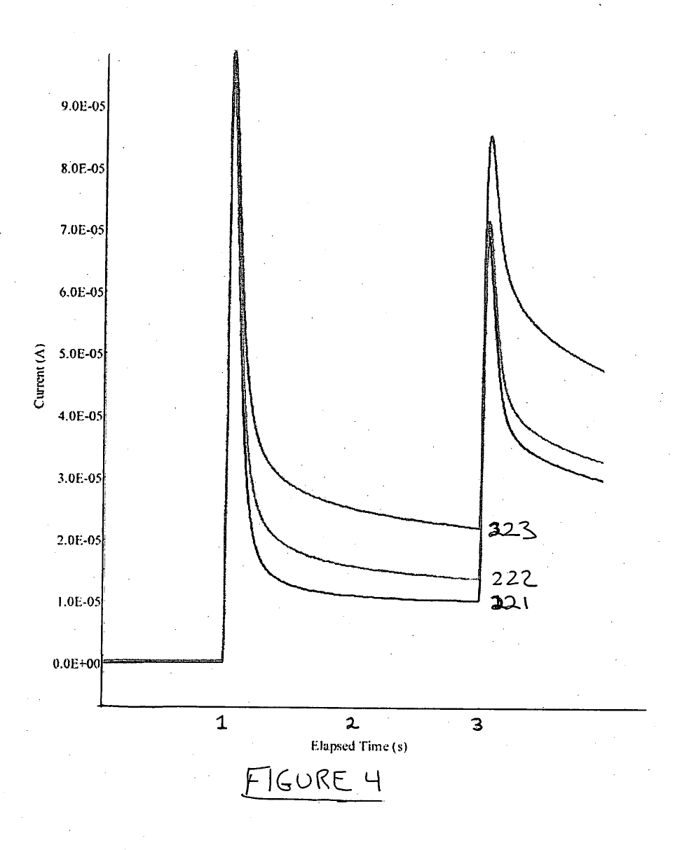

A prototype of the analytical electrode was tested using the solvent from Example 1. 150 mg of TNT sample in a paper packet was placed at a distance of 60 cm from the sensor element, i.e., approximately 10 cm from the end of the air probe of the inventive device. The measurement was carried out in a DC multi-pulse regime. The results of the test are provided in FIG. 4.

The parameters of the multi-pulse regime are as follows:
bias potential (E_{b})=−0.6 V, (Ag/AgCl, 3,5 M KCl);
E₁=+0.3 V; E₂=0.0 V;
E₃=0.0 V;
E₄=−0.15 V; t₁=t₂=t₃=t₄=1.0 sec; (Model 263A, Princeton Applied Research).

The presence of TNT was tested according to the following steps:
1. Air was sampled through an air probe for 30 seconds using a vacuum pump having an output of 1.5 liter/minute;
2. The background current at the analytical electrode was measured, yielding curve 221;
3. A first test curve 222 was generated using the above-mentioned explosive sample of TNT, by measuring the current at the analytical electrode as a function of time;
4. The explosive material trapped on the inner surface (made of polytetrafluoroethylene) of the sampling tube was then washed off with 5-7 ml of the above-mentioned solvent for 10 seconds;
5. A second test curve 223 was then generated by measuring the current at the analytical electrode as a function of time.

The results show a small, 2 μA difference between the first test curve 222 and the background curve 221, while the difference between second test curve 223 and background curve 221 is 12 μA.

The above result indicates that a major proportion (in this case, about 80%) of the explosive material can be trapped on the reversibly-trapping surface of the air sampler. Direct dissolution accounted for only about 20% of the explosive material. Thus, while direct dissolution can be used in electrochemical detectors, the combination of surface trapping with direct dissolution extraction is considerably—typically 4-5 times—more effective.

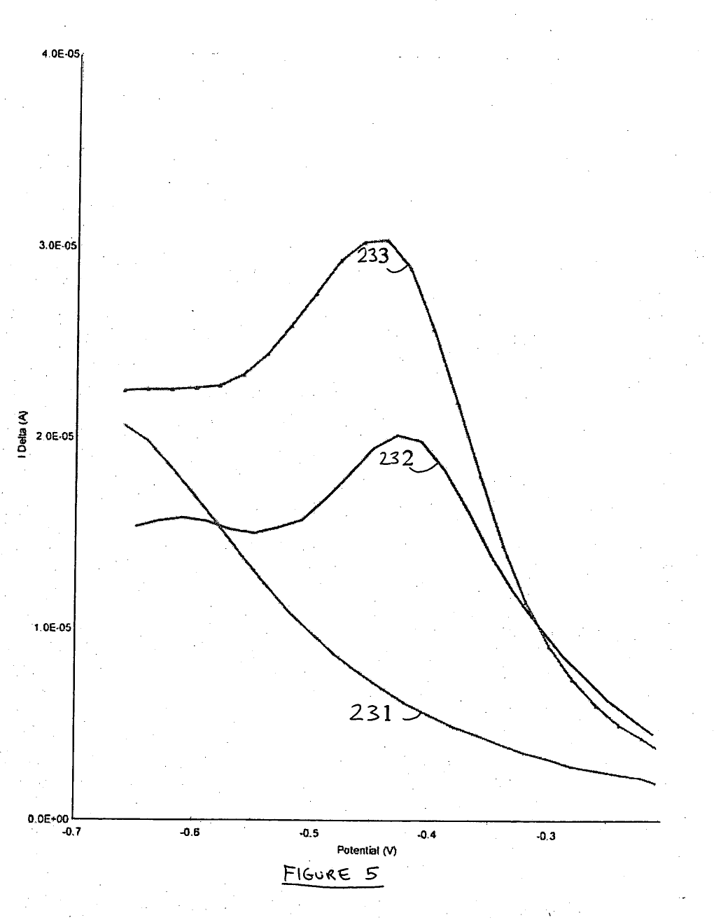

The dependence of the analytical signal of the modified electrode on the particular chemical modifier is illustrated in FIG. 5, where curve 231 is a background signal, curve 232 is produced by an electrode modified with a 4% aniline solution in DMSO, and curve 233 is produced by an electrode modified with a 4% solution of sulfanilamide in DMSO (as in Example 1). It is observed that both modified electrodes yield distinct current peaks at a potential of about −0.45V. It is further observed that for a given concentration of the explosive material, the electrode modified with sulfanilamide produces a much stronger detection signal than the electrode modified with aniline.

Without wishing to be limited by theory, the higher sensitivity attained with the sulfanilamide-modified electrode, relative to the aniline-modified electrode, is that the −NH₂ group in the sulfanilamide has is more positively charged. This produces a more stable bond between the negatively charged CO₂ group and the positively charged NH₂ group.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned -in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A portable device for detecting at least one explosive substance present in air, the device comprising:
(a) a mechanism for drawing an air sample into the device;
(b) a solid trapping material having a surface for trapping a portion of particles of an explosive material in said air sample;
(c) a collector for containing a solvent, said collector associated with said surface, said solvent for producing dissolved explosive material by:
(i) removing and dissolving said portion of particles from said surface, and
(ii) directly dissolving a remainder of said particles of said explosive material,
(d) an electrode unit, associated with said collector, for producing a signal corresponding to a presence of said dissolved explosive material, and
(e) circuitry for determining said presence of said dissolved explosive material based on said signal produced by said electrode unit.

2. The device of claim 1, wherein said trapping material is a reversibly trapping material.

3. The device of claim 2, wherein said trapping material includes a material selected from the group of materials consisting of polytetrafluoroethylene, cross-linked polyethylene, and polypropylene.

4. The device of claim 1, wherein said trap is associated with said collector by direct fluid communication.

5. The device of claim 1, further comprising an electrochemical mechanism, designed and configured to be in fluid communication with said collector, upon demand, for electrochemically regenerating said solvent.

6. The device of claim 5, further comprising a control device for enabling fluid communication between said electrochemical mechanism and said collector, upon demand.

7. The device of claim 2, wherein said surface of said solid trapping material has a reversibility factor (R), characterized by:
$$Reversibility Factor ( R ) = \frac{Weight of explosive material removed}{( Volume of Solvent ) \times ( Time )}$$
wherein said explosive material is trinitrotoluene, said solvent is ethylene glycol, said weight is measured in micrograms, said volume is measured in milliliters, and said time is measured in seconds,
and wherein R is at least 0.03.

8. The device of claim 7, wherein R is at least 0.05.

9. The device of claim 7, wherein R is at least 0.10.

10. The device of claim 1, wherein said solvent includes a solvent selected from the group of solvents consisting of ethylene glycol, propylene glycol, diethylene glycol dimethyl ether, triethylene glycol dimethyl ether, alcohols having a carbon chain length of 2 to 3, and mixtures thereof.

11. The device of claim 1, wherein said electrode unit includes an electrode material modified by treatment thereof with at least one chemical modifier that increases electron transfer kinetics of nitro-aromatic compounds.

12. The device of claim 11, wherein said at least one chemical modifier includes an aromatic organic compound.

13. The device of claim 11, wherein said at least one chemical modifier includes a compound selected from the group of compounds consisting of amine, sulfanilamide, amino-naphthalene and aromatic derivatives thereof, and a para-aminobenzenesulfonylamine having the structure NH₂—C₆H₄—SO₂—NR′R″, wherein said amine has R′ and R″ moieties selected from the group of radicals consisting of H, X, CH₃, CH₂X, and CHX₂, where H is hydrogen and X is a halogen or a halogen-containing moiety.

14. The device of claim 11, wherein said at least one chemical modifier includes a compound selected from the group of compounds consisting of amino-aromatic compounds, alkyl-aniline compounds, halide derivatives of alkyl aniline compounds and hydroxyl-aniline compounds.

15. The device of claim 11, wherein said at least one chemical modifier includes a compound selected from the group of compounds consisting of phenylene-diamine, diphenylene-diamine, and diphenylene-triamine.

16. The device of claim 11, wherein said at least one chemical modifier includes aniline.

17. The device of claim 1, wherein said electrode unit includes an electrode material selected from the group consisting of carbon and gold.

18. The device of claim 1, wherein said electrode unit includes an electrode material modified by treatment thereof with at least one chemical modifier, so as to produce, in a presence of said explosive material in said solvent, a current peak, said peak occurring within a potential range of 0.0 to minus 1.1 Volts.

19. The device of claim 18, wherein said current peak occurs within a potential range of minus 0.4 to minus 0.65 Volts.

20. The device of claim 18, wherein said current peak occurs within a potential range of minus 0.48 to minus 0.60 Volts.

21. The device of claim 1, wherein said circuitry includes a direct current pulse generator for registering at least 2 potential steps, wherein a duration of each of said potential steps is in a range of 0.1 to 2.5 seconds.

22. The device of claim 21, wherein said potential steps are in a cathode range that occurs minus 200 to minus 250 millivolts after a peak potential.

23. A portable device for detecting at least one explosive substance present in air, the device comprising:
(a) a mechanism for drawing an air sample into the device;
(b) a solid trapping material having a surface for trapping a portion of particles of an explosive material in said air sample;
(c) a collector for containing a solvent, said collector associated with said surface, said solvent for producing dissolved explosive material by removal of said portion of particles from said surface;
(d) a contacting mechanism for contacting said solvent with said surface so as to effect said removal;
(d) an electrode unit, associated with said collector, for producing a signal corresponding to a presence of said dissolved explosive material, and
(e) circuitry for determining said presence of said dissolved explosive material based on said signal produced by said electrode unit.

24. The device of claim 23, wherein said trapping material is a reversibly trapping material.

25. A method for detecting at least one explosive substance in air, the method comprising the steps of:
(a) providing a device including:
(i) a mechanism for drawing an air sample into the device;
(ii) a solid trapping material having a surface for trapping particles of an explosive material in said air sample;
(iii) a solvent for removing said particles from said surface and for dissolving said particles;
(iv) a collector for receiving said solvent, said collector associated with said surface, and
(v) an electrode unit, associated with said collector, for producing a signal corresponding to a presence of a dissolved explosive material;
(b) drawing an air sample into said device using said mechanism, so as to trap said particles on said surface;
(c) removing said particles from said surface into said solvent;
(d) dissolving said particles in said solvent to produce said dissolved explosive material, and
(e) producing said signal corresponding to said presence of said dissolved explosive material.

26. The method of claim 25, wherein said solid trapping material is a reversibly-trapping material.

27. The method of claim 26, the method further comprising:
(f) determining, automatically, said presence of said dissolved explosive material based on said signal produced by said electrode unit.

28. The method of claim 25, wherein said removing of said particles from said surface into said solvent is performed by automatically circulating said solvent, upon demand, so as to fluidly contact said surface.

29. The method of claim 25, the method further comprising:
(f) regenerating said solvent within said device.

30. The method of claim 29, wherein said regenerating is performed using regeneration electrodes.
